(19) United States
(12) Patent Application Publication
(10) Pub. No.: US 2025/0277803 A1
(43) Pub. Date: Sep. 4, 2025

(54) BLOOD-BASED LIPID BIOMARKER PANEL FOR PERSONALIZED RISK ASSESSMENT OF BREAST CANCER

(71) Applicant: Board of Regents, The University of Texas System, Austin, TX (US)

(72) Inventors: Samir HANASH, Houston, TX (US); Johannes F. FAHRMANN, Houston, TX (US); Ehsan IRAJIZAD, Houston, TX (US)

(21) Appl. No.: 19/206,876

(22) Filed: May 13, 2025

Related U.S. Application Data
(63) Continuation of application No. PCT/US2023/079958, filed on Nov. 16, 2023.
(60) Provisional application No. 63/384,162, filed on Nov. 17, 2022.

(51) Int. Cl. G01N 33/92

(52) U.S. Cl. CPC G01N 33/92; G01N 2405/08; G01N 2405/10; G01N 2560/00; G01N 2800/52

(57) ABSTRACT

A novel 11-lipid biomarker panel, consisting of or comprising at least one ceramide, at least one sphingomyelin, at least one glycosphingolipid, and at least one free fatty acid, capable of assessing the risk of breast cancer is described.

This application is a bypass continuation of International Application No. PCT/US2023/079958, filed Nov. 16, 2023, which claims the benefit of priority of U.S. Provisional Application No. 63/384,162, filed Nov. 17, 2022, the entirety of which is incorporated herein by reference.

Disclosed herein are methods and related kits for detection of breast cancer. Also provided are methods for treating a subject susceptible, or suspected of being susceptible, to breast cancer.

Screening mammography has been shown to decrease mortality from breast cancers. Currently, the United States Preventive Services Task Force (USPSTF) recommends biennial screening mammography for women of average risk aged 50 to 74 years with a ‘B’ recommendation. Screening mammography in women prior to age of 50 was given a ‘C’ recommendation, with decisions to be made at the individualized level.

However, mammography has come under considerable scrutiny on account of issues related to overscreening and overdiagnosis. Moreover, there are no tools currently available to guide personalized early detection of breast cancer, which has contributed to inconsistencies in national screening guidelines regarding the age at which mammography screening should start, the frequency of screening, and at what age mammogram screening should be stopped. Evidence has shown that metabolic syndrome characterized in part by obesity, hyperinsulinemia, and insulin resistance is associated with increased risk of breast cancer.

Accordingly, there is a need to establish biomarkers and/or a circulating biomarker metabolic profile to provide a personalized risk assessment for breast cancer and to tailor screening based on the individual's risk profile. A panel comprising or consisting of selected ceramides, sphingomyelins, glycosphingolipids, and free fatty acids, useful in methods of risk prediction and treatment of breast cancer, has been discovered. The lipid-based biomarker panel has utility for identifying women with ‘metabolic obesity’ who are at increased risk of breast cancer and would benefit from tailored screening.

### SUMMARY

Provided herein is a method of treatment of breast cancer in a subject having an elevated risk score or positive risk profile based on the subject's measured levels of at least one ceramide, at least one sphingomyelin, at least one glycosphingolipid, and at least one free fatty acid, wherein the elevated risk score or positive risk profile led to the subject's diagnosis with breast cancer, comprising administering a therapeutically effective amount of a treatment for breast cancer to the subject.

Also provided herein is a method of treatment of breast cancer, comprising:
a) identifying a subject with an elevated risk score or positive risk profile based on the subject's measured levels of at least one ceramide, at least one sphingomyelin, at least one glycosphingolipid, and at least one free fatty acid, wherein the elevated risk score or positive risk profile led to the subject's diagnosis with breast cancer; and
b) administering a therapeutically effective amount of a treatment for breast cancer to the subject.

Also provided herein is a method of determining the risk of a subject for breast cancer, comprising, in a biological sample obtained from the subject:
a) measuring the levels of at least one ceramide, at least one sphingomyelin, at least one glycosphingolipid, and at least one free fatty acid in the biological sample; and
b) classifying the subject as being at risk of breast cancer or not at risk of breast cancer based on the measured levels.

Also provided herein is a method of producing a risk profile of a subject for breast cancer, comprising, in a biological sample obtained from the subject:
a) measuring the levels of at least one ceramide, at least one sphingomyelin, at least one glycosphingolipid, and at least one free fatty acid in the biological sample; and
b) classifying the risk profile of the subject as being at risk of breast cancer (positive) or not at risk of breast cancer (negative) based on the measured levels.

Also provided herein is a method for calculating a subject's biomarker scores or risk score for breast cancer, comprising:
a) measuring the levels of at least one ceramide, at least one sphingomyelin, at least one glycosphingolipid, and at least one free fatty acid in a biological sample obtained from the subject; and
b) calculating the biomarker scores or risk score using the numerical values of the measured levels in a machine learning model.

Also provided herein is a method of risk stratification for a subject at risk for breast cancer, comprising, in a biological sample obtained from the subject:
a) measuring the levels of at least one ceramide, at least one sphingomyelin, at least one glycosphingolipid, and at least one free fatty acid in the biological sample; and
b) determining, by processor circuitry, the risk score for the subject, wherein the risk score is determined via a scoring function derived from metabolite profiles for biological samples taken from a plurality of individuals that were monitored for breast cancer.

Further methods and embodiments are disclosed below.

### BRIEF DESCRIPTION OF THE DRAWINGS

FIG. 1 depicts the predictive performance of the 11-marker lipid panel for distinguishing newly-diagnosed breast cancer patients from cancer free controls in the Discovery Cohort. Odds ratios per unit standard deviation increase is provided.

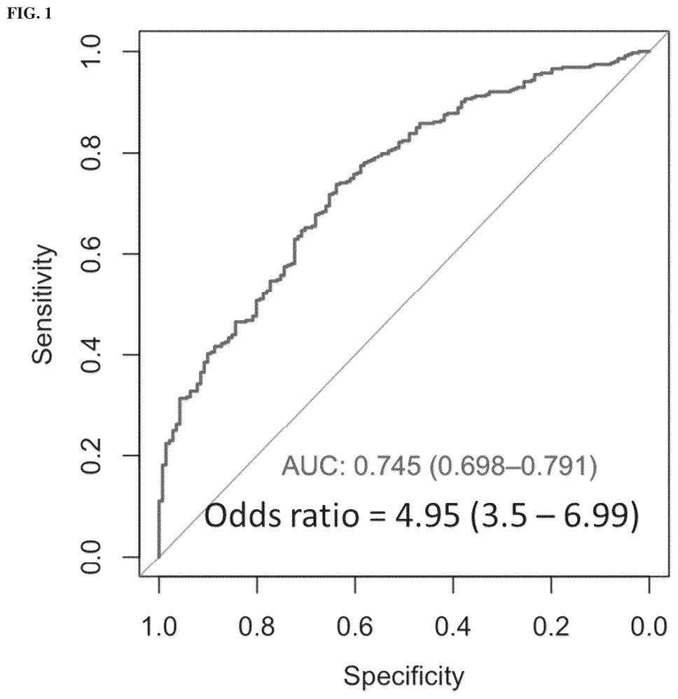

FIG. 2 depicts the predictive performance of the 11-marker lipid panel for distinguishing newly-diagnosed breast cancer patients from cancer free controls in the Validation Set. Odds ratios per unit standard deviation increase is provided.

FIG. 3 depicts the predictive performance of the 11-marker panel for distinguishing breast cancer cases from cancer-free controls among women with a body mass index (BMI)>=30 in the Discovery Cohort.

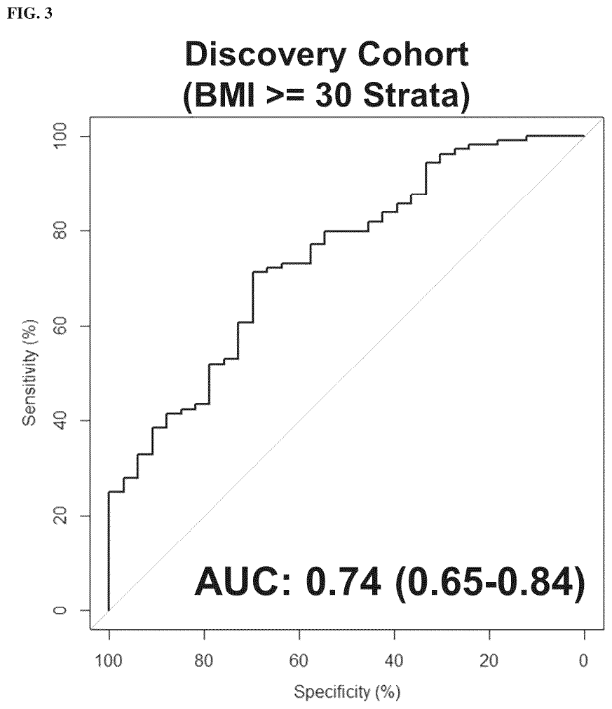

FIG. 4 depicts the predictive performance of the 11-marker panel for distinguishing breast cancer cases from cancer-free controls among women with a body mass index (BMI)>=30 in the Validation Cohort.

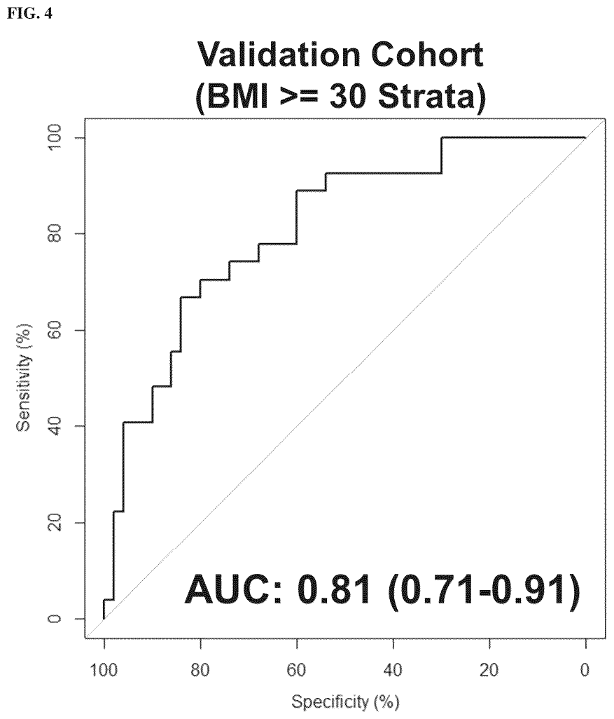

### DETAILED DESCRIPTION

Provided herein is a method of treatment of breast cancer in a patient having an elevated risk score or positive risk profile based on the subject's measured levels of at least one ceramide, at least one sphingomyelin, at least one glycosphingolipid, and at least one free fatty acid, wherein the elevated risk score or positive risk profile led to the subject's diagnosis with breast cancer, comprising administering a therapeutically effective amount of a treatment for breast cancer to the patient.

Also provided herein is a method of treatment of breast cancer, comprising:
a) identifying a patient with an elevated risk score or positive risk profile based on the subject's measured levels of at least one ceramide, at least one sphingomyelin, at least one glycosphingolipid, and at least one free fatty acid, wherein the elevated risk score or positive risk profile led to the subject's diagnosis with breast cancer; and
b) administering a therapeutically effective amount of a treatment for breast cancer to the patient.

Also provided herein is a method of determining the risk of a subject for breast cancer, comprising, in a biological sample obtained from the subject:
a) measuring the levels of at least one ceramide, at least one sphingomyelin, at least one glycosphingolipid, and at least one free fatty acid in the biological sample; and
b) classifying the subject as being at risk of breast cancer or not at risk of breast cancer based on the measured levels.

Also provided herein is a method of producing a risk profile of a subject for breast cancer, comprising, in a biological sample obtained from the subject:
a) measuring the levels of at least one ceramide, at least one sphingomyelin, at least one glycosphingolipid, and at least one free fatty acid in the biological sample; and
b) classifying the risk profile of the subject as being at risk of breast cancer (positive) or not at risk of breast cancer (negative) based on the measured levels.

Also provided herein is a method for calculating a subject's biomarker scores or risk score for breast cancer, comprising:
a) measuring the levels of at least one ceramide, at least one sphingomyelin, at least one glycosphingolipid, and at least one free fatty acid in a biological sample obtained from the patient; and
b) calculating the biomarker scores or risk score using the numerical values of the measured levels in a machine learning model.

Also provided herein is a method of risk stratification for a patient at risk for breast cancer, comprising, in a biological sample obtained from the patient:
a) measuring the levels of at least one ceramide, at least one sphingomyelin, at least one glycosphingolipid, and at least one free fatty acid in the biological sample; and
b) determining, by processor circuitry, the risk score for the patient, wherein the risk score is determined via a scoring function derived from metabolite profiles for biological samples taken from a plurality of individuals that were monitored for breast cancer.

Also provided herein is a method of treatment of breast cancer in a patient having an elevated risk score or positive risk profile based on the subject's measured levels of, or a method of determining the risk of a subject for, or producing a risk profile of a subject for, or for calculating a subject's biomarker scores or risk score for breast cancer, comprising, in a biological sample obtained from the subject, measuring the levels of, and classifying the subject as being at risk of breast cancer or not at risk of breast cancer based on the measured levels of one or more of ceramide (34:1), ceramide (42:2), sphingomyelin (33:1), sphingomyelin (34:1), sphingomyelin (42:2), Galα1-4Galβ1-4Glcβ-ceramide (42:2), NeuAcα2-3Galβ1-4Glcβ-ceramide (d18:1/16:0), palmitic acid, linoleic acid, and arachidonic acid.

In some embodiments, the machine learning model is a deep learning model (DLM).

In some embodiments, wherein the DLM comprises an artificial neural network which has at least one hidden layer and at least one node in each layer.

In some embodiments, at least one ceramide is chosen from ceramide (d18:2/16:0), ceramide (34:1), and ceramide (42:2).

In some embodiments, the at least one ceramide is three ceramides.

In some embodiments, the three ceramides are ceramide (d18:2/16:0), ceramide (34:1), and ceramide (42:2).

In some embodiments, ceramide (d18:2/16:0) has a mass-to-charge ratio between 515 and 565.

In some embodiments, ceramide (d18:2/16:0) has a mass-to-charge ratio of about 518.49.

In some embodiments, ceramide (34:1) has a mass-to-charge ratio between 515 and 565.

In some embodiments, ceramide (34:1) has a mass-to-charge ratio of about 520.51.

In some embodiments, ceramide (34:1) is chosen from ceramide (d14:1/20:0), ceramide (d16:1/18:0), and ceramide (d18:1/16:0).

In some embodiments, ceramide (42:2) has a mass-to-charge ratio between 625 and 675.

In some embodiments, ceramide (42:2) has a mass-to-charge ratio of about 670.61.

In some embodiments, ceramide (42:2) is chosen from ceramide (d18:1/24:1) and ceramide (d18:2/24:0).

In some embodiments, at least one sphingomyelin is chosen from sphingomyelin (33:1), sphingomyelin (34:1), and sphingomyelin (42:2).

In some embodiments, the at least one sphingomyelin is three sphingomyelins.

In some embodiments, the three sphingomyelins are sphingomyelin (33:1), sphingomyelin (34:1), and sphingomyelin (42:2).

In some embodiments, sphingomyelin (33:1) has a mass-to-charge ratio between 665 and 715.

In some embodiments, sphingomyelin (33:1) has a mass-to-charge ratio of about 687.55.

In some embodiments, sphingomyelin (33:1) is chosen from sphingomyelin (d16:1/17:0) and sphingomyelin (d18:1/15:0).

In some embodiments, sphingomyelin (34:1) has a mass-to-charge ratio between 680 and 730.

In some embodiments, sphingomyelin (34:1) has a mass-to-charge ratio of about 703.58.

In some embodiments, sphingomyelin (34:1) is chosen from sphingomyelin (d16:1/18:0), sphingomyelin (d17:1/17:0), and sphingomyelin (d18:1/16:0).

In some embodiments, sphingomyelin (42:2) has a mass-to-charge ratio between 790 and 840.

In some embodiments, sphingomyelin (42:2) has a mass-to-charge ratio of about 813.69.

In some embodiments, sphingomyelin (42:2) is chosen from sphingomyelin (d18:1/24:1) and sphingomyelin (d18:2/24:0).

In some embodiments, at least one glycosphingolipid is chosen from Galα1-4Galβ1-4Glcβ-ceramide (42:2) and NeuAcα2-3Galβ1-4Glcβ-ceramide (d18:1/16:0).

In some embodiments, the at least one glycosphingolipid is two glycosphingolipids.

In some embodiments, the two glycosphingolipids are Galα1-4Galβ1-4Glcβ-ceramide (42:2) and NeuAcα2-3Galβ1-4Glcβ-ceramide (d18:1/16:0).

In some embodiments, Galα1-4Galβ1-4Glcβ-ceramide (42:2) has a mass-to-charge ratio between 1115 and 1200.

In some embodiments, Galα1-4Galβ1-4Glcβ-ceramide (42:2) has a mass-to-charge ratio of about 1178.78.

In some embodiments, NeuAcα2-3Galβ1-4Glcβ-ceramide (d18:1/16:0) has a mass-to-charge ratio between 1130 and 1200.

In some embodiments, NeuAcα2-3Galβ1-4Glcβ-ceramide (d18:1/16:0) has a mass-to-charge ratio of about 1151.71.

In some embodiments, the at least one free fatty acid is chosen from palmitic acid, linoleic acid, and arachidonic acid.

In some embodiments, the at least one free fatty acid is three free fatty acids.

In some embodiments, the three free fatty acids are palmitic acid, linoleic acid, and arachidonic acid.

In some embodiments, the breast cancer is hormone-receptor (HR) positive.

In some embodiments, the breast cancer is human epidermal growth factor receptor 2 (HER2) positive.

In some embodiments, the breast cancer is triple-negative breast cancer (TNBC).

In some embodiments, each of the at least one ceramide, at least one sphingomyelin, at least one glycosphingolipid, and at least one free fatty acid generates a detectable signal.

In some embodiments, the detectable signals are detectable by a spectrometric method.

In some embodiments, the spectrometric method is chosen from UV-visible spectroscopy, mass spectroscopy, nuclear magnetic resonance (NMR) spectroscopy, proton NMR spectroscopy, nuclear magnetic resonance (NMR) spectrometry, gas chromatography, mass spectrometry (GC-MS), liquid chromatography-mass spectrometry (LC-MS), correlation spectroscopy (COSY), nuclear Overhauser effect spectroscopy (NOESY), rotating-frame nuclear Overhauser effect spectroscopy (ROESY), time-of-flight LC-MS (LC-TOF-MS), liquid chromatography-tandem mass spectrometry (LC-MS/MS), and capillary electrophoresis-mass spectrometry.

In some embodiments, the spectrometric method is mass spectrometry.

In some embodiments, the mass spectrometry is LC-TOF-MS.

In some embodiments, the treatment is chosen from surgery, chemotherapy, immunotherapy, radiation therapy, targeted therapy, or a combination thereof.

In some embodiments, the calculated biomarker scores, risk score, or risk profile are/is based on sensitivity and specificity values that corresponds to the risk threshold of the subject for breast cancer.

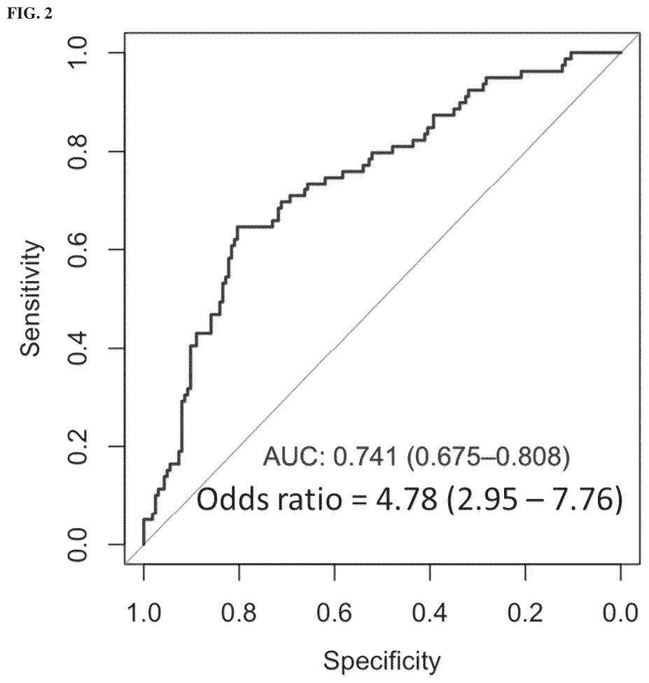

In some embodiments, the risk profile has sensitivity and specificity values that do not differ substantially from the curve in FIG. 2.

In some embodiments, the sensitivity and specificity values differ by less than 10%.

In some embodiments, the sensitivity and specificity values differ by less than 5%.

In some embodiments, the sensitivity and specificity values differ by less than 1%.

In some embodiments, the method further comprises assigning the patient to an appropriate risk group based on the calculated risk score.

In some embodiments, there are at least two risk groups.

In some embodiments, the risk score is measured against a given threshold value that represents the absolute risk of developing breast cancer over the next five years.

In some embodiments, the threshold value is greater than 0.001, or 0.1%.

In some embodiments, the threshold value is between 0.005 and 0.1, or 0.5% and 10%.

In some embodiments, the threshold value is about 0.01, or 1%.

In some embodiments, the threshold value is 0.01, or 1%.

In some embodiments, the risk score exceeds the threshold value and the patient is classified as being at risk for breast cancer.

In some embodiments, the risk score is below the threshold value and the patient is classified as being not at risk for breast cancer.

In some embodiments, the patient is subsequently designated for breast cancer screening.

In some embodiments, the screening is chosen from endoscopic ultrasound, magnetic resonance imaging (MRI), and computed topography (CT) scans.

In some embodiments, the screening is chosen from magnetic resonance imaging (MRI) and mammogram.

In some embodiments, the screening is performed annually.

In some embodiments, the screening is performed semi-annually

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value and the range which would be included by rounding up or down to that figure as well, taking into account significant figures, and the range which would encompass the recited value plus or minus 20%.

As used herein, the term “breast cancer” means a malignant neoplasm of the breast or pectoral area characterized by the abnormal proliferation of cells, the growth of which cells exceeds and is uncoordinated with that of the normal tissues around it.

As used herein, the term “subject” or “patient” as used herein refers to a mammal, preferably a human, for whom a classification as breast cancer-positive or breast cancer-negative is desired, and for whom further treatment can be provided.

As used herein, a “reference patient” or “reference group” refers to a group of patients or subjects to which a test sample from a patient suspected of having or being susceptible to breast cancer may be compared. In some embodiments, such a comparison may be used to determine whether the test subject has breast cancer. A reference patient or group may serve as a control for testing or diagnostic purposes. As described herein, a reference patient or group may be a sample obtained from a single patient, or may represent a group of samples, such as a pooled group of samples.

As used herein, “healthy” refers to an individual having healthy breast or pectoral tissue. A healthy patient or subject has no symptoms of breast cancer or other malignant growth of the breast area. In some embodiments, a healthy patient or subject may be used as a reference patient for comparison to diseased or suspected diseased samples for determination of breast cancer in a patient or a group of patients.

As used herein, “treating,” “treatment,” and the like means the administration of therapy to an individual who already manifests at least one symptom of a disease or condition or who has previously manifested at least one symptom of a disease or condition. For example, “treating” can include alleviating, abating, or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder. As related to the present disclosure, the term may also mean the administration of pharmacological substances or formulations, or the performance of non-pharmacological methods including, but not limited to, radiation therapy and surgery. Pharmacological substances as used herein may include, but are not limited to, chemotherapeutics that are established in the art, such as Gemcitabine (GEMZAR), 5-fluorouracil (5-FU), Ixabepilone (IXEMPRA), albumin-bound paclitaxel (ABRAXANE), capecitabine (XELODA), cisplatin, paclitaxel (TAXOL), docetaxel (TAXOTERE), and doxorubicin (ADRIAMYCIN). Pharmacological substances may include substances used in immunotherapy, such as checkpoint inhibitors. Treatment may include a multiplicity of pharmacological substances, or a multiplicity of treatment methods, including, but not limited to, surgery and chemotherapy.

As used herein, the term “machine learning model” refers to an analytical model generated by a machine learning algorithm or set of algorithms from a previously unseen dataset (or “training data”) that is validated in a subsequent test dataset and capable of recognizing patterns and making predictions in newly-presented specimen datasets. In some embodiments, the machine learning model is a deep learning model (DLM). In some embodiments, the machine learning model is a logistic regression. In some embodiments, the machine learning model is a LASSO regularization.

In some embodiments, the DLM comprises an artificial neural network. In some embodiments, the DLM has at least one hidden layer. In some embodiments, the DLM has at least one node in each layer. In some embodiments, the DLM has between one and five hidden layers and between 1 and 64 nodes in each layer. In some embodiments, the DLM has two hidden layers and 64 nodes in each layer.

As used herein, the term “regression” refers to a statistical method that can assign a predictive value for an underlying characteristic of a sample based on an observable trait (or set of observable traits) of said sample. In some embodiments, the characteristic is not directly observable. For example, the regression methods used herein can link a qualitative or quantitative outcome of a particular biomarker test, or set of biomarker tests, on a certain subject, to a probability that said subject is breast cancer-positive.

As used herein, the term “logistic regression” refers to a regression method in which the assignment of a prediction from the model can have one of several allowed discrete values. For example, the logistic regression models used herein can assign a prediction, for a certain subject, of either breast cancer-positive or breast cancer-negative.

As used herein, “amount” or “level” refers to a typically quantifiable measurement for a biomarker described herein, wherein the measurement enables comparison of the marker between samples and/or to control samples. In some embodiments, an amount or level is quantifiable and refers to the levels of a particular marker in a biological sample (e.g., blood, serum, urine, etc.), as determined by laboratory methods or tests such as an immunoassay, (e.g., antibodies), mass spectrometry, or liquid chromatography. In some embodiments, a marker may be present in the sample in an increased amount, or in a decreased amount. Marker comparisons may be based on direct measurement of the levels of a biomarker described herein, (e.g., through protein quantification or gene expression analysis) or may be based on measurement of e.g., reporter molecules, biomarker-receptor complexes, biomarker-relay-receptor complexes, or the like.

As used herein, the term “elevated” refers to a biomarker level or risk score in a given subject that is greater relative to the same biomarker level or model score in a given set of healthy patients or subjects.

As used herein, the term “biomarker score” refers to a numerical score for a given biomarker measured in a sample from a subject. The biomarker score is calculated by normalizing or weighting the measured level using a fixed coefficient as prescribed by the statistical method for a given biomarker panel. Biomarker scores are used as components in calculating a risk score for the subject. Elevated biomarker scores will carry more weight in risk score calculations and can indicate a higher risk for breast cancer for the subject.

As used herein, the term “risk score” refers to a single numerical value that indicates an asymptomatic human subject's risk for breast cancer as compared to the known prevalence in the disease cohort. The risk score is calculated through adding together the parameters of a statistical method derived from the subject for a given biomarker panel, which may take the form of biomarker scores, statistical model scores, or model constants. A higher risk score correlates to a higher risk for breast cancer in the subject. The risk score is empirically derived and will change depending on the data, cohort of the subject population, type of cancer, biomarkers chosen, occupational and environmental factors, and so on. In certain embodiments, the risk score as calculated for the human subject is the summation of the biomarker scores obtained from the subject. In certain embodiments, the risk score as calculated for the human subject is the summation of the biomarker scores obtained from the subject and one or more additional model constants. In certain embodiments, the risk score as calculated for a human subject is the summation of the biomarker scores obtained for the subject, normalized scores from one or more additional statistical models based on risk factors for the subject, and one or more additional model constants.

As used herein, the term “risk profile” refers to an assessment of a subject's risk score compared to those of a plurality of patients assessed using the same model, in which the patient is placed into an appropriate risk group based on a given score threshold. The score threshold is empirically derived and will change depending on the data, cohort of the subject population, type of cancer, biomarkers chosen, occupational and environmental factors, and so on. In certain embodiments, the subject's risk score exceeds the score threshold and their risk profile classifies them as being at risk for breast cancer (“positive”). In certain embodiments, the subject's risk profile is lower than the score threshold and classifies them as not being at risk for breast cancer (“negative”). In some embodiments, the score threshold is 0.001, or 0.1%, or greater. In some embodiments, the score threshold is 0.005, or 0.5%, or greater. In some embodiments, the score threshold is 0.01, or 1%, or greater. In some embodiments, the score threshold is 0.05, or 5%, or greater. In some embodiments, the score threshold is 0.1, or 10%, or greater.

As used herein, the term “cutoff point” refers to a mathematical value associated with a specific statistical method that can be used to assign a classification of breast cancer-positive or breast cancer-negative to a subject, based on said subject's biomarker score.

As used herein, when a numerical value above or below a cutoff value “is characteristic of breast cancer,” what is meant is that the subject, analysis of whose sample yielded the value, either has breast cancer or is at risk for breast cancer.

As used herein, the term “classification” refers to the assignment of a subject as either breast cancer-positive or breast cancer-negative, based on the result of the risk score or biomarker scores that is/are obtained for said subject.

As used herein, the term “breast cancer-positive” refers to an indication that a subject is predicted as susceptible to breast cancer, based on the results of the outcome of the methods of the disclosure.

As used herein, the term “breast cancer-negative” refers to an indication that a subject is predicted as not susceptible to breast cancer, based on the results of the outcome of the methods of the disclosure.

As used herein, the term “Wilcoxon rank sum test,” also known as the Mann-Whitney U test, Mann-Whitney-Wilcoxon test, or Wilcoxon-Mann-Whitney test, refers to a specific statistical method used for comparison of two populations. For example, the test can be used herein to link an observable trait, in particular a biomarker level, to the absence or presence of breast cancer in subjects of a certain population.

As used herein, the term “ROC” refers to receiver operating characteristic, which is a graphical plot used herein to gauge the performance of a certain diagnostic method at various cutoff points. A ROC plot can be constructed from the fraction of true positives and false positives at various cutoff points.

As used herein, the term “AUC” refers to the area under the curve of the ROC plot. AUC can be used to estimate the predictive power of a certain diagnostic test. Generally, a larger AUC corresponds to increasing predictive power, with decreasing frequency of prediction errors. Possible values of AUC range from 0.5 to 1.0, with the latter value being characteristic of an error-free prediction method.

As used herein, the term “p-value” or “p” refers to the probability that the distributions of biomarker scores for breast cancer-positive and breast cancer-negative subjects are identical in the context of a Wilcoxon rank sum test. Generally, a p-value close to zero indicates that a particular statistical method will have high predictive power in classifying a subject.

As used herein, the term “CI” refers to a confidence interval, i.e., an interval in which a certain value can be predicted to lie with a certain level of confidence. As used herein, the term “95% CI” refers to an interval in which a certain value can be predicted to lie with a 95% level of confidence.

As used herein, the term “11-marker lipid panel” refers to a panel of 11 lipid biomarkers, which includes ceramide (d18:2/16:0), ceramide (34:1), ceramide (42:2), sphingomyelin (33:1), sphingomyelin (34:1), sphingomyelin (42:2), Galα1-4Galβ1-4Glcβ-ceramide (42:2), NeuAcα2-3Galβ1-4Glcβ-ceramide (d18:1/16:0), palmitic acid, linoleic acid, and arachidonic acid. In some embodiments, the 11-marker lipid panel may be evaluated in combination with additional biomarkers or statistical models to enhance the prediction of breast cancer in biological samples from patients suspected as being at risk for breast cancer.

In some embodiments, using markers ceramide (d18:2/16:0), ceramide (34:1), ceramide (42:2), sphingomyelin (33:1), sphingomyelin (34:1), sphingomyelin (42:2), Galα1-4Galβ1-4Glcβ-ceramide (42:2), NeuAcα2-3Galβ1-4Glcβ-ceramide (d18:1/16:0), palmitic acid, linoleic acid, and arachidonic acid together as a panel may have an AUC (95% CI) of 0.65 or greater, including about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

As used herein, the term “sensitivity” refers to, in the context of various biochemical assays, the ability of an assay to correctly identify those with a disease (i.e., the true positive rate). By comparison, as used herein, the term “specificity” refers to, in the context of various biochemical assays, the ability of an assay to correctly identify those without the disease (i.e., the true negative rate). Sensitivity and specificity are statistical measures of the performance of a binary classification test (i.e., classification function). Sensitivity quantifies the avoiding of false negatives, and specificity does the same for false positives.

As used herein, “fixed coefficients” or “fixed model coefficients” refers to a statistical method of standardizing coefficients in order to allow comparison of the relative importance of each coefficient in a regression model. In some embodiments, fixed coefficients involve using the same beta-coefficients from a logistic regression model to yield a risk score for the developed combination rule, which is ultimately used to make a clinical decision based on a decision threshold(s).

As used herein, a “sample” refers to a test substance to be tested for the presence of, and levels or concentrations thereof, of a biomarker as described herein. A sample may be any substance appropriate in accordance with the present disclosure, including, but not limited to, blood, blood serum, blood plasma, or any part thereof.

As used herein, a “metabolite” refers to small molecules that are intermediates and/or products of cellular metabolism. Metabolites may perform a variety of functions in a cell, for example, structural, signaling, stimulatory and/or inhibitory effects on enzymes. In some embodiments, a metabolite may be a non-protein, plasma-derived metabolite marker, including, but not limited to, ceramides, sphingomyelins, sphingolipids, glycosphingolipids, and free fatty acids.

The phrase “therapeutically effective” is intended to qualify the amount of active ingredients used in the treatment of a disease or disorder or on the effecting of a clinical endpoint.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

### EXAMPLES

The invention is further illustrated by the following examples.

### Example 1: Patient Cohorts

Unbiased metabolomics profiling was conducted on an initial Development Set of plasmas collected from 353 newly-diagnosed breast cancer cases and 141 controls. An independent Test Set for validation of the model was conducted on 79 breast cancer cases and 163 controls. The performance of the model was evaluated among body mass index (BMI) strata (≥30 or <30 kg/m2) using a nested case: control matched design.

TABLE 1
Training and test cohorts

| Case/Control Status | |
|---|---|
| Development Set | Test Set |

| | Cases | Non-Cases | Cases | Non-Cases |
|---|---|---|---|---|
| # of Subjects | 353 | 141 | 79 | 163 |

### Example 2: Metabolomic Analysis

### Sample Extraction

Pre-aliquoted serum or plasma samples (10 μL) were extracted with 30 μL of LCMS grade 2-propanol (ThermoFisher) in a 96-well microplate (Eppendorf). Plates were heat sealed, vortexed for 5 min at 750 rpm, and centrifuged at 2000×g for 10 minutes at room temperature. The supernatant (10 μL) was carefully transferred to a 96-well plate, leaving behind the precipitated protein. The supernatant was further diluted with 90 μL of 1:3:2 100 mM ammonium formate, pH 3 (Fischer Scientific): LCMS grade acetonitrile (ThermoFisher): LCMS grade 2-propanol (ThermoFisher) and transferred to a 384-well microplate (Eppendorf) for lipids analysis using LCMS.

### Untargeted Metabolomic Analyses

Untargeted metabolomics analysis was conducted on a Waters Acquity™ UPLC system coupled to a Xevo G2-XS quadrupole time-of-flight (qTOF) mass spectrometer. Chromatographic separation was performed using a C18 (Acquity™ UPLC HSS T3, 100 Å, 1.8 μm, 2.1×100 mm, Water Corporation, Milford, U.S.A) column at 55° C. The quaternary solvent system mobile phases were (A) water, (B) acetonitrile, (C) 2-propanol and (D) 500 mM ammonium formate, pH 3. A starting elution gradient of 20% A, 30% B, 49% C and 1% D was linearly changed to 4% A, 14% B, 81% C and 1% D for 4.5 min, followed by isocratic elution at 4% A, 14% B, 81% C and 1% D for 2.1 min and column equilibration with initial conditions for 1.4 min.

### Mass Spectrometry Data Acquisition

Mass spectrometry data was acquired using ‘sensitivity’ mode in positive and negative electrospray ionization mode within 100-2000 Da. For the electrospray acquisition, the capillary voltage was set at 1.5 kV (positive), 3.0 kV (negative), sample cone voltage 30 V, source temperature at 120° C., cone gas flow 50 L/h and desolvation gas flow rate of 800 L/h with a scan time of 0.5 sec in continuum mode. Leucine Enkephalin; 556.2771 Da (positive) and 554.2615 Da (negative) was used for lockspray correction and scans were performed at 0.5 sec. The injection volume for each sample was 3 μL. The acquisition was carried out with instrument auto gain control to optimize instrument sensitivity over the sample acquisition time.

### Data Processing

LC-MS and LC-MSe data may be processed using Progenesis QI (Nonlinear, Waters). Peak picking and retention time alignment of LC-MS and MSe data may be performed using Progenesis QI software (Nonlinear, Waters). Data processing and peak annotations may be performed using an in-house automated pipeline. Annotations may be determined by matching accurate mass and retention times using customized libraries created from authentic standards and by matching experimental tandem mass spectrometry data against the NIST MSMS, LipidBlast or HMDB v3 theoretical fragmentations; for complex lipids retention time patterns characteristic of lipid subclasses may also considered. To correct for injection order drift, each feature may be normalized using data from repeat injections of quality control samples collected every 10 injections throughout the run sequence. Measurement data may be smoothed by Locally Weighted Scatterplot Smoothing (LOESS) signal correction (QC-RLSC) as previously described. Values may be reported as ratios relative to the median of historical quality control reference samples run with every analytical batch for the given analyte. To account for any potential batch effects, metabolite readouts may be median-centered and values may be log₁₀-transformed.

### Example 3: Lipid Database

A total of 11 lipid biomarkers encompassing lipid subclasses with known pro-inflammatory and tumor promoting roles were detected and quantified across all specimens, including six sphingolipids (three ceramides, three sphingomyelins), two glycosphingolipids, and three free fatty acids.

TABLE 2
Lipids comprising the 11-Marker Lipid Panel

| | | Retention | mass-to- | | |
|---|---|---|---|---|---|
| | | Time | charge | | Ionization |
| Name | Domain | (RT) | ratio (m/z) | Adduct | Mode |
| Ceramide | Sphingolipid | 3.56 | 518.4912 | [M − | ESI_Positive |
| (d18:2/16:0) | | | | H2O + H]+ | |
| Ceramide(34:1) | Sphingolipid | 3.94 | 520.5085 | [M − | ESI_Positive |
| | | | | H2O + H]+ | |
| Ceramide(42:2) | Sphingolipid | 5.06 | 670.6108 | [M + Na]+ | ESI_Positive |
| Sphingomyelin | Sphingolipid | 3.26 | 687.5451 | [M − H]− | ESI_Negative |
| (33:1) | | | | | |
| Sphingomyelin | Sphingolipid | 3.23 | 703.5792 | [M + H]+ | ESI_Positive |
| (34:1) | | | | | |
| Sphingomyelin | Sphingolipid | 4.49 | 813.6852 | [M + H]+ | ESI_Positive |
| (42:2) | | | | | |
| Gala1-4Galβ1- | Glycosphingolipid | 4.33 | 1178.7783 | [M + FA − | ESI_Negative |
| 4Glcβ- | | | | H]− | |
| Ceramide(42:2) | | | | | |
| NeuAca2-3Galβ1- | Glycosphingolipid | 2.53 | 1151.7062 | [M − H]− | ESI_Negative |
| 4Glcβ- | | | | | |
| Ceramide(d18:1/16:0 | | | | | |
| Palmitic acid | Free Fatty Acid | 1.96 | 255.2327 | [M − H]− | ESI_Negative |
| Linoleic acid | Free Fatty Acid | 1.66 | 279.2327 | [M − H]− | ESI_Negative |
| Arachidonic acid | Free Fatty Acid | 1.58 | 303.2321 | [M − H]− | ESI_Negative |

### ESI: Electrospray Ionization

### Example 4: Model Building and Testing

Different learning algorithms may be evaluated including deep learning (fully connected feed forward network), gradient boosting machine and auto-machine learning, iterative random forest, LASSO regularization, and logistic regression models. Deep learning, extreme gradient boost, and auto machine learning algorithms may be performed in the h2o package in R. Iterative random forests may be run using the iRF package in R. To further evaluate model stability in accordance with PCS framework, data perturbations (e.g. via random selection and replacement) may be introduced to the Development Set and the performance re-assessed. Based on AUC, a deep learning model (DLM) with two hidden layers and 64 nodes per layer was developed in the Development Set and validated in the set-aside Test Set.

### Performance of the 11-Marker Lipid Panel Among Non-Obese and Obese Women

The predictive performance estimates of the individual ceramide metabolites yielded AUCs (95% CI) between 0.56-0.62 in the Development Set and 0.58-0.64 in the Validation Set, with p-values ranging from 0.001-0.028. The individual sphingomyelin metabolites yielded AUCs (95% CI) between 0.55-0.57 in the Development Set and 0.59-0.60 in the Validation Set, with p-values ranging from 0.012-0.044. The individual glycosphingolipid metabolites yielded AUCs (95% CI) between 0.57-0.58 in the Development Set and 0.61-0.64 in the Validation Set, with p-values ranging from 0.000-0.012. The individual free fatty acid metabolites yielded AUCs (95% CI) between 0.56-0.59 in the Development Set and 0.63-0.69 in the Validation Set, with p-values ranging from 0.000-0.036.

The 11-marker lipid biomarker panel yielded an AUC of 0.75 (95% CI: 0.70-0.79) for distinguishing breast cancer cases from controls in the Development Set. Predictive performance of the lipid panel was comparable when stratifying cases into hormone-receptor (HR) positive, HER2-positive/HR negative, and triple-negative breast cancer subtypes. The biomarker panel had an AUC of 0.74 (95% CI: 0.68-0.81) in the independent Validation Set. Using a case: control matched design, the performance of the model among body mass index (BMI) strata (≥30 or <30 kg/m2) was additionally evaluated, which revealed that the predictive performance of the panel was most pronounced among obese subjects (BMI≥30) with an AUC of 0.81 (95% CI: 0.71-0.91) in the Test Set.

TABLE 3
Performance of the 11-Marker Lipid Panel for distinguishing
breast cancer cases, stratified by subtype from
controls in the Training Cohort.

| Subtype | Case, N | Control, N | AUC | 95% CI |
|---|---|---|---|---|
| HR+ | 238 | 141 | 0.7293 | 0.6782 to 0.7804 |
| HER2+ | 16 | 141 | 0.6959 | 0.5513 to 0.8405 |
| Non-TNBC | 254 | 141 | 0.7272 | 0.6767 to 0.7777 |
| TNBC | 85 | 141 | 0.7322 | 0.6648 to 0.7997 |

HR+: Hormone receptor positive
HER2+: human epidermal growth factor receptor 2 positive
TNBC: triple-negative breast cancer
AUC: Area Under the Receiver Operating Characteristic Curve
CI: confidence interval

TABLE 4
Performance of the 11-Marker Lipid Panel among
non-obese and obese women in Training Cohort

| Strata | Case, N | Control, N | AUC | 95% CI |
|---|---|---|---|---|
| All | 353 | 141 | 0.7316 | 0.6845 to 0.7787 |
| Non-Obese (BMI <30) | 243 | 106 | 0.7379 | 0.6829 to 0.7929 |
| Obese (BMI 30+) | 104 | 33 | 0.7401 | 0.6457 to 0.8345 |

| | Non-Obese (BMI <30) | | Obese (BMI ≥30) | |
|---|---|---|---|---|
| Specificity | Sensitivity | | Sensitivity | |
| Threshold (%) | (%) | 95% CI | (%) | 95% CI |
| 99 | 16.87 | 12.69% to 22.09% | 25.00 | 17.67% to 34.11% |
| 95 | 27.16 | 21.96% to 33.08% | 32.69 | 23.57% to 41.19% |
| 90 | 37.86 | 32.00% to 44.10% | 38.46 | 29.68% to 48.06% |
| 85 | 42.39 | 36.34% to 48.67% | 41.35 | 32.35% to 50.95% |
| 80 | 51.85 | 45.59% to 58.06% | 43.27 | 34.16% to 52.86% |

BMI: Body-mass index

TABLE 5
Performance of the 11-Marker Lipid Panel among
non-obese and obese women in Validation Cohort

| Strata | Case, N | Control, N | AUC | 95% CI |
|---|---|---|---|---|
| All | 79 | 160 | 0.7448 | 0.6787 to 0.8109 |
| Non-Obese (BMI <30) | 51 | 109 | 0.7068 | 0.6194 to 0.7941 |
| Obese (BMI 30+) | 26 | 50 | 0.8092 | 0.7091 to 0.9093 |

| | Non-Obese (BMI <30) | | Obese (BMI ≥30) | |
|---|---|---|---|---|
| Specificity | Sensitivity | 95% | Sensitivity | 95% |
| Threshold (%) | (%) | CI | (%) | CI |
| 99 | 7.84 | 3.092% to 18.50% | 3.85 | 0.1973% to 18.89% |
| 95 | 17.65 | 9.572% to 30.25% | 42.31 | 25.54% to 61.05% |
| 90 | 25.49 | 15.55% to 38.87% | 50.00 | 32.06% to 67.94% |
| 85 | 39.22 | 27.03% to 52.91% | 53.85 | 35.46% to 71.24% |
| 80 | 54.90 | 41.38% to 67.73% | 69.23 | 50.01% to 83.50% |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of treatment of breast cancer in a subject having an elevated risk score or positive risk profile based on the subject's measured levels of at least one ceramide, at least one sphingomyelin, at least one glycosphingolipid, and at least one free fatty acid, wherein the elevated risk score or positive risk profile led to the subject's diagnosis with breast cancer, comprising administering a therapeutically effective amount of a treatment for breast cancer to the subject.

2. A method of treatment of breast cancer, comprising:
a) identifying a subject with an elevated risk score or positive risk profile based on the subject's measured levels of at least one ceramide, at least one sphingomyelin, at least one glycosphingolipid, and at least one free fatty acid, wherein the elevated risk score or positive risk profile led to the subject's diagnosis with breast cancer; and
b) administering a therapeutically effective amount of a treatment for breast cancer to the subject.

3. A method of determining the risk of a subject for breast cancer, comprising, in a biological sample obtained from the subject:
a) measuring the levels of at least one ceramide, at least one sphingomyelin, at least one glycosphingolipid, and at least one free fatty acid in the biological sample; and
b) classifying the subject as being at risk of breast cancer or not at risk of breast cancer based on the measured levels.

4. A method of producing a risk profile of a subject for breast cancer, comprising, in a biological sample obtained from the subject:
a) measuring the levels of at least one ceramide, at least one sphingomyelin, at least one glycosphingolipid, and at least one free fatty acid in the biological sample; and
b) classifying the risk profile of the subject as being at risk of breast cancer (positive) or not at risk of breast cancer (negative) based on the measured levels.

5. A method for calculating a subject's biomarker scores or risk score for breast cancer, comprising:
a) measuring the levels of at least one ceramide, at least one sphingomyelin, at least one glycosphingolipid, and at least one free fatty acid in a biological sample obtained from the subject; and
b) calculating the biomarker scores or risk score using the numerical values of the measured levels in a machine learning model.

6. A method of risk stratification for a subject at risk for breast cancer, comprising, in a biological sample obtained from the subject:
a) measuring the levels of at least one ceramide, at least one sphingomyelin, at least one glycosphingolipid, and at least one free fatty acid in the biological sample; and
b) determining, by processor circuitry, the risk score for the subject, wherein the risk score is determined via a scoring function derived from metabolite profiles for biological samples taken from a plurality of individuals that were monitored for breast cancer.

7. The method of claim 5, wherein the machine learning model is a deep learning model (DLM).

8. The method of claim 5, wherein the DLM comprises an artificial neural network which has two hidden layers and 64 nodes in each layer.

9. The method of any one of claims 1-8, wherein at least one ceramide is chosen from ceramide (d18:2/16:0), ceramide (34:1), and ceramide (42:2).

10. The method of any one of claims 1-8, wherein the at least one ceramide is three ceramides.

11. The method of claim 10, wherein the three ceramides are ceramide (d18:2/16:0), ceramide (34:1), and ceramide (42:2).

12. The method of claim 11, wherein ceramide (d18:2/16:0) has a mass-to-charge ratio between 515 and 565.

13. The method of claim 12, wherein ceramide (d18:2/16:0) has a mass-to-charge ratio of about 518.49.

14. The method of claim 11, wherein ceramide (34:1) has a mass-to-charge ratio between 515 and 565.

15. The method of claim 14, wherein ceramide (34:1) has a mass-to-charge ratio of about 520.51.

16. The method of claim 11, wherein ceramide (42:2) has a mass-to-charge ratio between 625 and 675.

17. The method of claim 16, wherein ceramide (42:2) has a mass-to-charge ratio of about 670.61.

18. The method of any one of claims 1-17, wherein at least one sphingomyelin is chosen from sphingomyelin (33:1), sphingomyelin (34:1), and sphingomyelin (42:2).

19. The method of any one of claims 1-17, wherein the at least one sphingomyelin is three sphingomyelins.

20. The method of claim 19, wherein the three sphingomyelins are sphingomyelin (33:1), sphingomyelin (34:1), and sphingomyelin (42:2).

21. The method of claim 20, wherein sphingomyelin (33:1) has a mass-to-charge ratio between 665 and 715.

22. The method of claim 21, wherein sphingomyelin (33:1) has a mass-to-charge ratio of about 687.55.

23. The method of claim 20, wherein sphingomyelin (34:1) has a mass-to-charge ratio between 680 and 730.

24. The method of claim 23, wherein sphingomyelin (34:1) has a mass-to-charge ratio of about 703.58.

25. The method of claim 20, wherein sphingomyelin (42:2) has a mass-to-charge ratio between 790 and 840.

26. The method of claim 25, wherein sphingomyelin (42:2) has a mass-to-charge ratio of about 813.69.

27. The method of any one of claims 1-26, wherein at least one glycosphingolipid is chosen from Galα1-4Galβ1-4Glcβ-ceramide (42:2) and NeuAcα2-3Galβ1-4Glcβ-ceramide (d18:1/16:0).

28. The method of any one of claims 1-26, wherein the at least one glycosphingolipid is two glycosphingolipids.

29. The method of claim 28, wherein the two glycosphingolipids are Galα1-4Galβ1-4Glcβ-ceramide (42:2) and NeuAcα2-3Galβ1-4Glcβ-ceramide (d18:1/16:0).

30. The method of claim 29, wherein Galα1-4Galβ1-4Glcβ-ceramide (42:2) has a mass-to-charge ratio between 1115 and 1200.

31. The method of claim 30, wherein Galα1-4Galβ1-4Glcβ-ceramide (42:2) has a mass-to-charge ratio of about 1178.78.

32. The method of claim 29, wherein NeuAcα2-3Galβ1-4Glcβ-ceramide (d18:1/16:0) has a mass-to-charge ratio between 1130 and 1200.

33. The method of claim 32, wherein NeuAcα2-3Galβ1-4Glcβ-ceramide (d18:1/16:0) has a mass-to-charge ratio of about 1151.71.

34. The method of any one of claims 1-33, wherein the at least one free fatty acid is chosen from palmitic acid, linoleic acid, and arachidonic acid.

35. The method of any one of claims 1-33, wherein the at least one free fatty acid is three free fatty acids.

36. The method of claim 35, wherein the three free fatty acids are palmitic acid, linoleic acid, and arachidonic acid.

37. The method of any one of claims 1-36, wherein the breast cancer is hormone-receptor (HR) positive.

38. The method of any one of claims 1-36, wherein the breast cancer is human epidermal growth factor receptor 2 (HER2) positive.

39. The method of any one of claims 1-36, wherein the breast cancer is triple-negative breast cancer (TNBC).

40. The method of any preceding claim, wherein each of the at least one ceramide, at least one sphingomyelin, at least one glycosphingolipid, and at least one free fatty acid generates a detectable signal.

41. The method of claim 40, wherein the detectable signals are detectable by a spectrometric method.

42. The method of claim 41, wherein the spectrometric method is chosen from UV-visible spectroscopy, mass spectroscopy, nuclear magnetic resonance (NMR) spectroscopy, proton NMR spectroscopy, nuclear magnetic resonance (NMR) spectrometry, gas chromatography, mass spectrometry (GC-MS), liquid chromatography-mass spectrometry (LC-MS), correlation spectroscopy (COSY), nuclear Overhauser effect spectroscopy (NOESY), rotating-frame nuclear Overhauser effect spectroscopy (ROESY), time-of-flight LC-MS (LC-TOF-MS), liquid chromatography-tandem mass spectrometry (LC-MS/MS), and capillary electrophoresis-mass spectrometry.

43. The method of claim 42, wherein the spectrometric method is mass spectrometry.

44. The method of claim 43, wherein the mass spectrometry is LC-TOF-MS.

45. The method of any one of claims 1-44, wherein the treatment is chosen from surgery, chemotherapy, immunotherapy, radiation therapy, targeted therapy, or a combination thereof.

46. The method of any one of claims 1-8, wherein the calculated biomarker scores, risk score, or risk profile are/is based on sensitivity and specificity values that corresponds to the risk threshold of the subject for breast cancer.

47. The method of claim 46, wherein the risk profile has sensitivity and specificity values that do not differ substantially from the curve in FIG. 2.

48. The method of claim 47, wherein the sensitivity and specificity values differ by less than 10%.

49. The method of claim 48, wherein the sensitivity and specificity values differ by less than 5%.

50. The method of claim 49, wherein the sensitivity and specificity values differ by less than 1%.

51. The method as recited in any previous claim, further comprising assigning the subject to an appropriate risk group based on the calculated risk score.

52. The method of claim 51, wherein there are at least two risk groups.

53. The method as recited in any previous claim, wherein the risk score is measured against a given threshold value that represents the absolute risk of developing breast cancer over the next five years.

54. The method of claim 53, wherein the threshold value is greater than 0.001, or 0.1%.

55. The method of claim 54, wherein the threshold value is between 0.005 and 0.1, or 0.5% and 10%.

56. The method of claim 55, wherein the threshold value is about 0.01, or 1%.

57. The method of claim 56, wherein the threshold value is 0.01, or 1%.

58. The method of any one of claims 53-57, wherein the risk score exceeds the threshold value and the subject is classified as being at risk for breast cancer.

59. The method of any one of claims 53-57, wherein the risk score is below the threshold value and the subject is classified as being not at risk for breast cancer.

60. The method of claim 58, wherein the subject is subsequently designated for breast cancer screening.

61. The method of claim 60, wherein the screening is chosen from magnetic resonance imaging (MRI) and mammogram.

62. The method of claim 60, wherein the screening is performed annually.

63. The method of claim 60, wherein the screening is performed semi-annually.
